(19) United States
(12) Patent Application Publication
(10) Pub. No.: US 2023/0404983 A1
(43) Pub. Date: Dec. 21, 2023

(54) COMPOSITIONS, DEVICES AND METHODS FOR TREATING OBSESSIVE-COMPULSIVE DISORDER

(71) Applicant: CENTRE FOR DIGESTIVE DISEASES, Five Dock (AU)

(72) Inventor: Thomas Julius BORODY, Five Dock (AU)

(21) Appl. No.: 18/242,276

(22) Filed: Sep. 5, 2023

Related U.S. Application Data
(63) Continuation of application No. 17/208,272, filed on Mar. 22, 2021, now abandoned.
Application No. 17/208,272 is a continuation of application No. 16/471,475, filed on Jun. 19, 2019, now abandoned.
(60) Provisional application No. 62/552,517, filed on Aug. 31, 2017.

(51) Int. Cl. A61K 31/437; A61P 25/00; A61K 9/00; A61K 31/4164; A61K 38/14

(52) U.S. Cl. CPC A61K 31/437; A61P 25/00; A61K 9/0056; A61K 31/4164; A61K 38/14

(57) ABSTRACT

In alternative embodiments, provided are pharmaceutical compositions and methods for treating, ameliorating, reversing and/or preventing (acting as a prophylaxis) an Obsessive-Compulsive Disorder (OCD), with or without an accompanying autism or an autism spectrum disorder (ASD), e.g., a regressive autism. In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole, secnidazole or a combination thereof. As there are various molecular forms of rifaximins, all these are useful and used in methods and compositions as provided herein

### FIELD

This invention generally relates to medicine and gastroenterology, pharmacology and microbiology. In alternative embodiments, provided are pharmaceutical compositions and methods for treating, ameliorating, reversing and/or preventing (acting as a prophylaxis) an Obsessive-Compulsive Disorder (OCD), with or without an accompanying autism or an autism spectrum disorder (ASD), e.g., a regressive autism. In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole, secnidazole or a combination thereof. As there are various molecular forms of rifaximins, all these are useful and used in methods and compositions as provided herein.

### BACKGROUND

There are conditions that are clinically characterised by a super-infected stool but where the bacterial super-infection can be difficult to detect. These pose a medical challenge with treatment. Among such conditions are Obsessive-Compulsive Disorder (OCD), Irritable Bowel Syndrome, Colitis, Autism/Autism Spectrum Disorder (ASD), amongst others. In OCD, ASD and other listed and non-listed conditions here, some researchers have identified unusual Clostridia, e.g. Clostridium boltae and Desulfovibrios. These may be an underlying pathogenic mechanism, and may cause the development and manufacture within the colonic flora of neurotoxins. Such neurotoxins can ultimately enter the peripheral circulation in a child or adult that has acquired such an infection during a course of antibiotics in its youth—and the toxins attach themselves to various areas in the brain such as Broca's area, association areas in the parietal cortex and visual areas in the occipital cortex. When those parts of the brain are affected by the neurotoxins, neurotransmission is inhibited and the children and adults can develop the classic symptoms of OCD with excessive thoughts (obsessions) that lead to repetitive behaviours (compulsions) in the presence or absence of Autism.

OCD is characterised by unreasonable thoughts and fears (obsessions) that lead to compulsive behaviours. OCD often centers on themes such as a fear of germs or the need to arrange objects in a specific manner. Symptoms usually begin gradually and vary throughout life, and in the presence of ASD may include repetitive movements, and at times loss of learned speech an inability to see mother's eyes, or loss of eye contact. Treatment includes talk therapy, medication or both.

Given that the origin of the neurotoxins is the bowel flora—considered to be the body's largest organ, albeit a ‘virtual organ’ since it does not carry human DNA cells—this super-infection has been a target for therapy to modify the OCD and often Autism. Sandler et al. (Journal of Child Neurology; July 2000; 15, 7) reported that treatment of such children with vancomycin may suppress the production of these neurotoxins, resulting in reversal of behavioural changes and progress to normality within a few weeks of treatment on vancomycin, which is not absorbable. Other antibiotics can be used for this condition, for example, Rodakis reported that a child's autistic symptoms reversed almost completely when treated with an amoxycillin preparation (Rodakis J., Microbial Ecology in Health & Disease 2015; 26: 26382). Similar results for autistic symptoms have been observed with rifaximin alone or combined with other anti-Clostridium agents such a vancomycin and nitroimidazoles.

### SUMMARY OF INVENTION

In alternative embodiments, provided are compositions, including formulations, pharmaceutical compositions, foods, feeds, supplements, products of manufacture, and the like, for treating, ameliorating, reversing and/or preventing (acting as a prophylaxis) an Obsessive-Compulsive Disorder (OCD) comprising use or inclusion of a rifaximin or equivalent thereof as the sole antibiotic or antibacterial, or a rixafimin and another antimicrobial or antibiotic agent, for example, vancomycin, metronidazole, tinidazole, a secnidazole, or a combination thereof. In alternative embodiments, the Obsessive-Compulsive Disorder (OCD) is accompanied by an autism or an autism spectrum disorder (ASD), optionally a regressive autism setback-type autism, or an acquired autistic syndrome.

In alternative embodiments, provided are methods and compositions for treating, ameliorating, reversing and/or preventing (acting as a prophylaxis) an Obsessive-Compulsive Disorder (OCD) to an individual in need thereof, comprising administering to the individual in need thereof:
(a) a formulation, a pharmaceutical preparation or a pharmaceutical composition comprising or consisting of a rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, or
(b) a formulation, a pharmaceutical preparation or a pharmaceutical composition comprising or consisting of a rifaximin, a polymorphic form of a rifaximin, or rifaximin equivalent thereof, (optionally a XIFAXAN™, XIFAXANTA™ or NORMIX™) and a formulation, a pharmaceutical preparation or a pharmaceutical composition comprising at least one additional antimicrobial or antibiotic agent,

wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

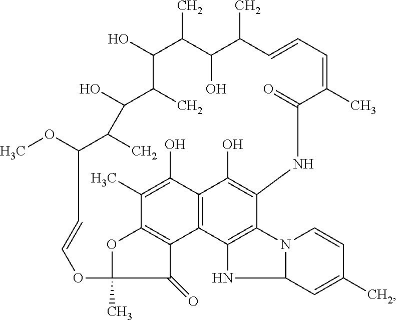

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/−0.2 and 18.33 degree+/−0.2, 2 theta.

In alternative embodiments, the OCD further comprises or is associated with an autism or an autism spectrum disorder (ASD), optionally an autistic disorder, a pervasive developmental disorder not otherwise specified (PDD-NOS), and/or an Asperger syndrome.

In alternative embodiments, the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.

In alternative embodiments, the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.

In alternative embodiments, the at least one additional antimicrobial or antibiotic agent comprises a vancomycin, a metronidazole (optionally Flagyl™, Metro™), a tinidazole (optionally Fasigyn™, Simplotan™, Tindamax™), an ornidazole (optionally XYNOR™), a secnidazole (optionally Flagentyl™, Sindose™, Secnil™), or a combination thereof.

In alternative embodiments, the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an ornidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.

In alternative embodiments, the method comprises administering the individual in need thereof a formulation, a pharmaceutical preparation or a pharmaceutical composition comprising or consisting of a rifaximin, a polymorphic form of a rifaximin, or rifaximin equivalent thereof, (optionally a XIFAXAN™, XIFAXANTA™ or NORMIX™) and a formulation, a pharmaceutical preparation or a pharmaceutical composition comprising at least one additional antimicrobial or antibiotic agent. The formulation, a pharmaceutical preparation or a pharmaceutical composition comprising or consisting of rifaximin, a polymorphic form of a rifaximin, or rifaximin equivalent thereof, (optionally a XIFAXAN™, XIFAXANTA™ or NORMIX™) and the formulation, a pharmaceutical preparation or a pharmaceutical composition comprising at least one additional antimicrobial or antibiotic agent may be administered simultaneously or may be administered sequentially.

In alternative embodiments, the individual exhibits at least an about 5% to 10% reduction in OCD symptom severity after administration of the formulation, pharmaceutical preparation or pharmaceutical composition to the individual in need thereof as compared to before initiating the administration.

In alternative embodiments, the at least about 5% to 10% reduction in OCD symptom severity is achieved after about 1 to 2 or more weeks, or after about 1 to 2 months, of initiating the administration.

In alternative embodiments, the at least about 5% to 10% reduction in OCD symptom severity is maintained for at least about 4 to 8 weeks after discontinuing the administration.

In alternative embodiments, the formulation, the pharmaceutical or the pharmaceutical preparation is formulated as a chewable delivery vehicle, a gum, a gummy, a candy, a lozenge, an ice cream or an ice, or a yogurt.

In alternative embodiments, a unit dosage is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.

In alternative embodiments, a daily dosage is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.

In alternative embodiments, a unit dosage is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

In alternative embodiments, a daily dosage is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.

In alternative embodiments, a daily dosage is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week, and optionally this “ramping up” or increasing of dosages continues for about a month, or about 2, 3, 4, 5 or 6 months or more or for about a year, or until symptoms of OCD significantly diminish or abate, or significantly diminish or abate without need for administration of the formulation, the pharmaceutical or the pharmaceutical preparation.

In alternative embodiments, the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

In alternative embodiments, the formulation, the pharmaceutical or the pharmaceutical preparation further comprises a preservative, a benzoic acid or a potassium sorbate.

In alternative embodiments, the formulation, the pharmaceutical or the pharmaceutical preparation further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.

In alternative embodiments, the formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch.

In alternative embodiments, the formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

In alternative embodiments, the formulation, the pharmaceutical or the pharmaceutical preparation further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, the formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

In alternative embodiments, the formulation, the pharmaceutical or the pharmaceutical preparation is contained in a delivery vehicle, product of manufacture, container, syringe, device or bag.

In alternative embodiments, the formulation, the pharmaceutical or the pharmaceutical preparation is manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In alternative embodiments, the method comprises administering to the individual in need thereof rifaximin and a nitroimidazole. In further embodiments, the method comprises administering to the individual in need thereof rifaximin and tinidazole. In further embodiments, the method comprises administering to the individual in need thereof rifaximin, vancomycin, and a nitroimidazole. In further embodiments, the method comprises administering to the individual in need thereof rifaximin, vancomycin, and metronidazole.

The details of one or more embodiments of the invention are set forth in the accompanying description below. Other features, objects, and advantages of the invention will be apparent from the description and the claims.

In a first aspect, forms of the invention described herein include the following:
1. Use of:
   (a) a formulation, a pharmaceutical preparation or a pharmaceutical composition comprising or consisting of a rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, or
   (b) a formulation, a pharmaceutical preparation or a pharmaceutical composition comprising a rifaximin, a polymorphic form of a rifaximin, or rifaximin equivalent thereof, (optionally a XIFAXAN™, XIFAXANTA™ or NORMIX™) and at least one additional antimicrobial or antibiotic agent, or
   wherein optionally for (a) or (b) the rifaximin or rifaximin polymorphic form thereof or rifaximin equivalent comprises:
   (i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
   (ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

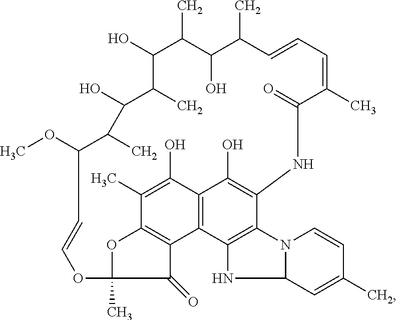

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/− and 18.33 degree+/−0.2, 2 theta;
in the manufacture of a medicament for treating, ameliorating, reversing and/or preventing (acting as a prophylaxis) an Obsessive-Compulsive Disorder (OCD) in an individual in need thereof.
2. Use of rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof,
wherein optionally the rifaximin or rifaximin polymorphic form thereof or rifaximin equivalent comprises:
   (i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
   (ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

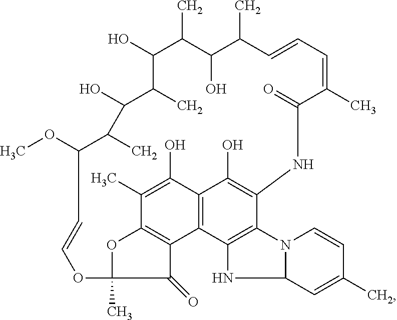

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/− and 18.33 degree+/−0.2, 2 theta;
in the manufacture of a medicament for treating, ameliorating, reversing and/or preventing (acting as a prophylaxis) an Obsessive-Compulsive Disorder (OCD) in an individual in need thereof, wherein said medicament is formulated for use in combination with at least one additional antimicrobial or antibiotic agent.
3. The use of form 2, wherein said medicament is formulated for sequential administration with the least one additional antimicrobial or antibiotic agent.
4. The use of form 2, wherein said medicament is formulated for simultaneous administration with the least one additional antimicrobial or antibiotic agent.
5. The use of any one of the preceding forms, wherein the OCD further comprises or is associated with an autism or an autism spectrum disorder (ASD), optionally an autistic disorder, a pervasive developmental disorder not otherwise specified (PDD-NOS), and/or an Asperger syndrome.
6. The use of any one of the preceding forms, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
7. The use of any one of the preceding forms, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.
8. The use of any one of the preceding forms, wherein the at least one additional antimicrobial or antibiotic agent comprises:
   an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
   an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
   a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
   an equivalent thereof or a combination thereof
9. The use of any one of the preceding forms wherein the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™), an ornidazole (optionally XYNOR™), a secnidazole (optionally FLAGENTYL™, SINDOSE™, SECNIL™), or a combination thereof
10. The use of any one of the preceding forms, wherein the individual exhibits at least an about 5% to 10% reduction in OCD symptom severity after administration of the medicament as compared to before initiating the administration.
11. The use of form 9, wherein the at least about 5% to 10% reduction in OCD symptom severity is achieved after about 1 to 2 or more weeks, or after about 1 to 2 months, of initiating the administration.
12. The use of form 9, wherein the at least about 5% to 10% reduction in OCD symptom severity is maintained for at least about 4 to 8 weeks after discontinuing the administration.
13. The use of any one of the preceding forms, wherein the medicament is formulated as a chewable delivery vehicle, a gum, a gummy, a candy, a lozenge, an ice cream or an ice, or a yogurt.
14. The use of any one of the preceding forms, wherein the medicament is formulated as a pediatric unit dosage, and optionally the unit dosage is between about 10 mg and 1100 mgm, or between about between about 40 mg and 4,000 mgm, or is about 10, 20, 30, 40, 50, 60, 70, 75, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.
15. The use of any one of the preceding forms, wherein the medicament is formulated for a daily dosage of about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.
16. The use of any one of the preceding forms, wherein a unit dosage of the medicament is formulated for administration (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
17. The use of any one of the preceding forms, wherein the medicament is formulated for a daily dosage of about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.
18. The use of any one of the preceding forms, wherein the medicament is formulated for daily dosage that is increased or “ramped up” every week, or every other week, by about 25, 50, 100, 125, 150, 175, 200, 225, 250, 275, 300, 325, 350, 375, 400, 425, 450, 475, 1500, 2000, 2500, 3000, 3500, 4000, or more or more mg per week, or every other week, and optionally this “ramping up” or increasing of dosages continues for about a month, about 6 months or about a year, or until symptoms of OCD significantly diminish or abate, or significantly diminish or abate without need for administration of the medicament.
19. The use of any one of the preceding forms, wherein the medicament further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
20. The use of any one of the preceding forms, wherein the medicament further comprises a preservative, a benzoic acid or a potassium sorbate.
21. The use of any one of the preceding forms, wherein the medicament further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.
22. The use of any one of the preceding forms, wherein the medicament further comprises, or has added to: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch.
23. The use of any one of the preceding forms, wherein the medicament further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
24. The use of any one of the preceding forms, wherein the medicament further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof
25. The use of any one of the preceding forms, wherein the medicament is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
26. The use of any one of the preceding forms, wherein the medicament is contained in a delivery vehicle, product of manufacture, container, syringe, device or bag.
27. The use of any one of the preceding forms, wherein the medicament is formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.
28. The use of any one of the preceding forms, wherein the medicament comprises rifaximin and a nitroimidazole.
29. The use of any one of the preceding forms, wherein the medicament comprises rifaximin and tinidazole.
30. The use of any one of the preceding forms, wherein the medicament comprises rifaximin, vancomycin, and metronidazole.

In a second aspect, forms of the invention described herein include the following:
1. A composition comprising or consisting of:
   (a) a rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, or
   (b) a rifaximin, a polymorphic form of a rifaximin, or rifaximin equivalent thereof, (optionally a XIFAXAN™, XIFAXANTA™ or NORMIX™) and at least one additional antimicrobial or antibiotic agent,
   wherein optionally for (a) or (b) the rifaximin or rifaximin polymorphic form thereof or rifaximin equivalent comprises:
   (i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
   (ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

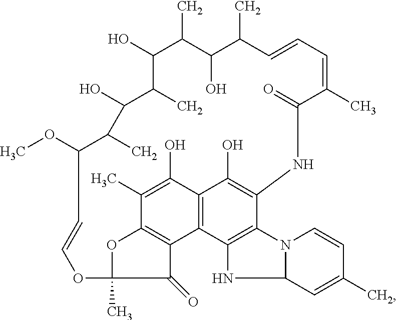

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/− and 18.33 degree+/−0.2, 2 theta; for use in treating, ameliorating, reversing and/or preventing (acting as a prophylaxis) an Obsessive-Compulsive Disorder (OCD) in an individual in need thereof.
2. A composition comprising a rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof,
wherein optionally the rifaximin or rifaximin polymorphic form thereof or rifaximin equivalent comprises:
   (i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
   (ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

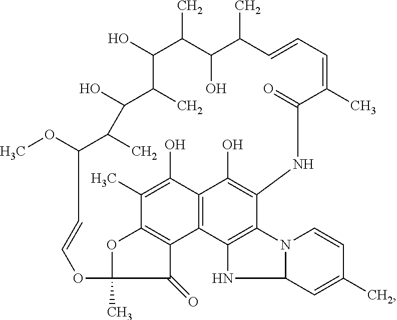

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/− and 18.33 degree+/−0.2, 2 theta;
for use in treating, ameliorating, reversing and/or preventing (acting as a prophylaxis) an Obsessive-Compulsive Disorder (OCD) in an individual in need thereof, wherein said use is in combination with at least one additional antimicrobial or antibiotic agent.
3. The composition of form 2, wherein in said use the at least one additional antimicrobial or antibiotic agent and the rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, are administered sequentially.
4. The composition of form 2, wherein in said use the at least one additional antimicrobial or antibiotic agent and the rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, are administered simultaneously.
5. The composition of any one of the preceding forms, wherein the OCD further comprises or is associated with an autism or an autism spectrum disorder (ASD), optionally an autistic disorder, a pervasive developmental disorder not otherwise specified (PDD-NOS), and/or an Asperger syndrome.
6. The composition of any one of the preceding forms, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
7. The composition of any one of the preceding forms, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.
8. The composition of any one of the preceding forms, wherein the at least one additional antimicrobial or antibiotic agent comprises:
   an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
   an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
   a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
   an equivalent thereof or a combination thereof.
9. The composition of any one of the preceding forms wherein the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™), an ornidazole (optionally XYNOR™), a secnidazole (optionally FLAGENTYL™, SINDOSE™, SECNIL™), or a combination thereof.
10. The composition of any one of the preceding forms, wherein the individual exhibits at least an about 5% to 10% reduction in OCD symptom severity after administration of the composition as compared to before initiating the administration.
11. The composition of form 9, wherein the at least about 5% to 10% reduction in OCD symptom severity is achieved after about 1 to 2 or more weeks, or after about 1 to 2 months, of initiating the administration.
12. The composition of form 9, wherein the at least about 5% to 10% reduction in OCD symptom severity is maintained for at least about 4 to 8 weeks after discontinuing the administration.
13. The composition of any one of the preceding forms, wherein the composition is formulated as a chewable delivery vehicle, a gum, a gummy, a candy, a lozenge, an ice cream or an ice, or a yogurt.
14. The composition of any one of the preceding forms, wherein the composition is formulated as a pediatric unit dosage, and optionally the unit dosage is between about 10 mg and 1100 mgm, or between about between about 40 mg and 4,000 mgm, or is about 10, 20, 30, 40, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.
15. The composition of any one of the preceding forms, wherein the composition is formulated for a daily dosage is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.
16. The composition of any one of the preceding forms, wherein the composition is formulated for a unit dosage set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
17. The composition of any one of the preceding forms, wherein the composition is formulated for a daily dosage of about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.
18. The composition of any one of the preceding forms, wherein the composition is formulated for a daily dosage that is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week, and optionally this “ramping up” or increasing of dosages continues for about a month, about 6 months or about a year, or until symptoms of OCD significantly diminish or abate, or significantly diminish or abate without need for administration of the formulation, the pharmaceutical or the pharmaceutical preparation.
19. The composition of any one of the preceding forms, further comprising a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
20. The composition of any one of the preceding forms, further comprising a preservative, a benzoic acid or a potassium sorbate.
21. The composition of any one of the preceding forms, further comprising: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.
22. The composition of any one of the preceding forms, further comprising: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch.
23. The composition of any one of the preceding forms, further comprising an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
24. The composition of any one of the preceding forms, further comprising: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof
25. The composition of any one of the preceding forms, wherein the composition is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
26. The composition of any one of the preceding forms, wherein the composition is contained in a delivery vehicle, product of manufacture, container, syringe, device or bag.
27. The composition of any one of the preceding forms, wherein the composition is formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.
28. The use of any one of the preceding forms, wherein the composition comprises rifaximin and a nitroimidazole.
29. The use of any one of the preceding forms, wherein the composition comprises rifaximin and tinidazole.
30. The use of any one of the preceding forms, wherein the composition comprises rifaximin, vancomycin, and metronidazole.

All publications, patents, patent applications cited herein are hereby expressly incorporated by reference for all purposes.

### DESCRIPTION OF EMBODIMENTS

In alternative embodiments, provided are pharmaceutical compositions and methods for treating, ameliorating, reversing and/or preventing (acting as a prophylaxis) Obsessive-Compulsive Disorder (OCD). In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole, an ornidazole, a secnidazole, or a combination thereof.

In alternative embodiments, antibiotics and antibacterials used to practice methods as provided herein are formulated and dosaged for oral administration as a powder, e.g., a lyophilised powder, which can be inserted into carriers, e.g., capsules, tablets, geltabs, and the like, e.g., for administration to autistic infants or children (or those suspected of developing Obsessive-Compulsive Disorder (OCD)) to ingest.

Because Obsessive-Compulsive Disorder (OCD) may present itself at the age of 2.5 years or above the children are unlikely to be able to swallow a capsule; thus, this provided are additional delivery vehicles, products of manufacture and devices to be combined with formulations as provided herein, e.g., powders such as lyophilised powders, e.g., lyophilised powder in a storage vehicle, e.g., capsules, geltabs and the like; for example, provided are delivery vehicles, products of manufacture and devices manufactured as a container, a kit, a package or a pack of a “device and capsule” together, e.g., operably associated such that the container, kit, package or a pack permits individuals, e.g., the very young children and the older children (and including disabled or handicapped individuals) to ingest the product, e.g., the lyophilised product, from the storage vehicle, e.g., capsules, geltabs and the like.

In alternative embodiments, the container, kit, a package or a pack provides the ability of any age child (or disabled or handicapped individual, or any individual) to ingest or swallow the product (e.g., a formulation, pharmaceutical preparation or pharmaceutical composition as provided herein) within the storage vehicle (e.g., capsule) by “draining”, e.g., by puncturing, crushing, twisting or turning the container by hand or a device, or otherwise opening, the storage vehicle using a puncturing, crushing or equivalent device (operably built into the container, kit, package or pack), or by hand motion, e.g., by twisting or hand turning (e.g., by hand) the container, and thus allowing passage or contact of the contents of the storage vehicle to enter or pass into an ingestible liquid or other edible substance (e.g., an ice cream or a yoghurt), which is also contained within the container, kit, package or pack, which can be initially (before the twisting or turning, puncturing, crushing or otherwise opening) in a separate compartment from the storage compartment. This twisting or turning, or puncturing, crushing or otherwise opening of the storage compartment and the passage or contact of the contents of the storage vehicle to the ingestible liquid effectively places the contents of the storage (e.g., a powder or freeze-dry comprised of or within a formulation, pharmaceutical preparation or pharmaceutical composition as provided herein) into the ingestible liquid or substance, which can be e.g., water, a milk, a yoghurt, an ice cream, a yogurt, a juice (e.g., a fruit juice, an apple juice), an apple sauce, or a masking drink. The container, kit, package or pack can be designed as an infant feeding bottle, e.g., comprising a nipple or teat for the very young.

In alternative embodiments, this simple twisting or turning, or puncturing or crushing device, allows the storage containers, e.g., geltabs or capsules, to be punctured and/or crushed or otherwise “opened”, allowing the contents of the storage container, (e.g., a powder or freeze-dry comprised of or within a formulation, pharmaceutical preparation or pharmaceutical composition as provided herein), to fall out in to the liquid or food compartment, e.g., to the bottom end of a device or straight into a bottle or a container held underneath or configured to be attached and underneath. For example, in this way a provider, e.g., the mother, can purchase a supply of storage containers, e.g., geltabs or capsules, convert them as needed into a powder capable of being mixed a liquid of her choice that the child will be ingesting.

In alternative embodiments, for those capable of swallowing tablets, capsules and the like, the storage containers, e.g., geltabs, tablets or capsules, are manufactured as enteric coated to bypass the acid of the stomach and bile of the duodenum, such that the storage containers, e.g., geltabs, tablets or capsules open (e.g., dissolve) in the jejunum or below.

In alternative embodiments, further provided are instructions for use, e.g., that when emptied into a drink, providers (e.g., the mothers of infants or children) are advised to choose a drink or food that has its own buffering capacity such as flavoured milk, chocolate milk, ice cream, yoghurt, ice blocks, frozen icicles, or simply milk, e.g., that is being fed to the infant or child by a bottle, e.g., a milk bottle, with a nipple or teat.

In alternative embodiments, storage containers, e.g., geltabs, tablets or capsules, or any formulation as provided herein, also comprises an antacid, e.g., a calcium carbonate, magnesium hydroxide, propylene glycol alginate and sodium alginate, or the combination of aluminium hydroxide with magnesium trisilicate, magnesium oxide or magnesium carbonate, so that when the storage container is punctured, crushed or otherwise opened and put into contact with the liquid, e.g., the feeding bottle, and ingested, there will be greater protection from acid damage. In alternative embodiments, methods and instructions further comprise the infant or child also being given an acid suppressant beforehand to permit more viable living bacteria to arrive in the colon.

In alternative embodiments, formulations, pharmaceuticals or pharmaceutical preparations as provided herein are formulated or manufactured as storage vehicles, e.g., tablets, geltabs, pills, capsules and the like; and in alternative embodiments, these storage vehicles are contained in, or contained in a kit with, or packaged with, or sold together with, a storage vehicle ‘cracking’, puncturing, or otherwise opening or releasing device (e.g., as a powder, e.g., as lyophilised material). These can be dispensed together, or configured together, or manufactured together, as a simple way of meeting the needs of both infants, the very young, older children and needful (e.g., handicapped) adults; e.g., as a powder, e.g., as lyophilised material, e.g., from their storage vehicles, e.g., as encapsulated formulations, pharmaceuticals or pharmaceutical preparations, thus permitting successful clinical administration on a frequent, e.g., bid, tid, or daily, basis for prolonged periods.

### Methods of Use and Applications of Devices and Compositions

In alternative embodiments, provided are compositions, devices and methods for treating, ameliorating, reversing and/or preventing (acting as a prophylaxis) Obsessive-Compulsive Disorder (OCD). In alternative embodiments, compositions and methods as provided herein can be used effectively for treating, ameliorating, reversing and/or preventing (acting as a prophylaxis) conditions associated with Obsessive-Compulsive Disorder (OCD), including: Rett Syndrome, various anxiety disorders as well as major depressive disorders, bipolar disorders, anorexia nervosa, bulimia nervosa, Tourette's syndrome, Attention Deficit Hyperactivity Disorder, Trichotillomania and Dermatillomania.

### Multicomponent Packaging

Provided are multi-component delivery systems, e.g., products of manufacture, comprising e.g., formulations, pharmaceutical preparations or pharmaceutical compositions used to practice methods as provided herein, e.g., formulated and dosaged for oral administration as a powder, e.g., a lyophilised powder, and another component, e.g., a liquid; these multi-component delivery systems, e.g., products of manufacture, can be designed or manufactured as described e.g., in U.S. Pat. Nos. 8,968,717; 8,931,665; 7,861,854; 7,018,089; 6,626,912; and, U.S. Pat. App. Pub nos. 2010/0034574; 2009/0180923; 20090232886; 2008/0160076; 2007/0087048; 2007/0036830; 2007/0074979; 2005/0205438; 2004/0089563.

### Packaging

Provided are compositions, including preparations, formulations and/or kits, comprising combinations of ingredients, as described herein. In alternative embodiments, these combinations can be mixed and administered together, or alternatively, they can be an individual member of a packaged combination of ingredients, e.g., a liquid component and a solid product component manufactured in a separate compartment, package, kit or container; e.g., where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a formulations, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of formulations, pharmaceutical preparations or pharmaceutical compositions as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb IL) using regular heat seal tooling. This alternative aspect, using heat seal tooling, can seal common types of thermoformed packaging.

In alternative embodiments, formulations, pharmaceutical preparations or pharmaceutical compositions are formulated, e.g., as a powder, e.g., as lyophilised material, e.g., a lyophilized encapsulated product, e.g., for practicing methods as provided herein, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of formulations, pharmaceutical preparations or pharmaceutical compositions as provided herein. Products or kits comprise an aqueous solution(s) which are dispensed (e.g., by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system gives tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic agent. This safeguards the therapeutic agent's efficacy by protecting each tablet, geltab or pill until it's taken; gives the product or kit portability, makes it easy to take a dose anytime or anywhere.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

A 16-year-old female patient with OCD that had suffered for many years with obsessive hand washing and household cleaning, and exhibited a behaviour of putting together knives and forks in a particular symmetrical order, was treated with the combination of rifaximin 500 mg bd and tinidazole 250 mg bd. The rifaximin dose was increased from 500 mg bd to 500 mg tds and the tinidazole was also increased after 2 weeks to 250 mg tds. Over the next 6 months the patient's obsessive hand washing, household cleaning and obsession with knife and fork placement was noted to be progressively reduced and ultimately ceased completely at around 7-8 months. The dose of tinidazole was then increased to 500 mg twice daily and the patient remained free of the OCD symptoms mentioned above for the next 7 months of follow-up to date.

### Example 2

A 22-year-old male with regressive ASD also had associated Obsessive Compulsive Disorder on presentation. The OCD was characterised by feeling a compulsion to bring his hands together as if in prayer numerous times per day. He would also hoard and hide clothing and other items such as cards. The patient possessed a vocabulary of only about 20 or 30 words when commenced on rifaximin 500 mg tds with vancomycin 250 mg tds, later adding metronidazole 200 mg bd. After 7 months of treatment his condition began to improve. Over the next 3 months (months 8-10) the patient's OCD activities progressively diminished and were only rarely seen by month 10. One year after commencing treatment the patient does not show any further evidence of OCD and his vocabulary as assessed by a speech pathologist has increased to around 50 words.

1. A method for treating, ameliorating, reversing and/or preventing (acting as a prophylaxis) an Obsessive-Compulsive Disorder (OCD) in an individual in need thereof, comprising administering to the individual in need thereof:
(a) a formulation, a pharmaceutical preparation or a pharmaceutical composition comprising or consisting of a rifaximin (optionally a XIFAXAN™, XIFAXANTA™ or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, or equivalents thereof or a mixture or a combination thereof, or
(b) a formulation, a pharmaceutical preparation or a pharmaceutical composition comprising or consisting of a rifaximin, a polymorphic form of a rifaximin, or rifaximin equivalent thereof, (optionally a XIFAXAN™, XIFAXANTA™ or NORMIX™) and a formulation, a pharmaceutical preparation or a pharmaceutical composition comprising at least one additional antimicrobial or antibiotic agent,
wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula: 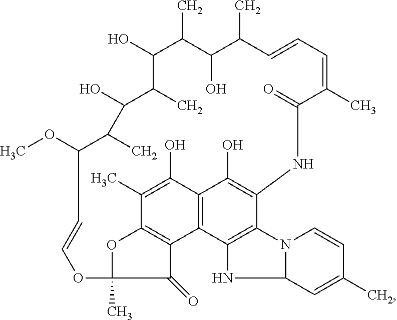
(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+/−0.2-18.33 degree+/−0.2, 2 theta, with maximum at about 7.75 degree+/−0.2 and in the range 14.54 degree+/−0.2 and 18.33 degree+/−0.2, 2 theta.

2. The method of claim 1, wherein the OCD further comprises or is associated with an autism or an autism spectrum disorder (ASD), optionally an autistic disorder, a pervasive developmental disorder not otherwise specified (PDD-NOS), and/or an Asperger syndrome.

3. The method of claim 1 or claim 2, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.

4. The method of any one of the preceding claims, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.

5. The method of any one of the preceding claims, wherein the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.

6. The method of any one of the preceding claims wherein the at least one additional antimicrobial or antibiotic agent comprises a vancomycin, a metronidazole (optionally Flagyl™, Metro™), a tinidazole (optionally Fasigyn™, Simplotan™, Tindamax™), an ornidazole (optionally XYNOR™), a secnidazole (optionally Flagentyl™, Sindose™, Secnil™), or a combination thereof.

7. The method of any one of the preceding claims, wherein the individual exhibits at least an about 5% to 10% reduction in OCD symptom severity after administration of the formulation, pharmaceutical preparation or pharmaceutical composition to the individual in need thereof as compared to before initiating the administration.

8. The method of claim 6, wherein the at least about 5% to 10% reduction in OCD symptom severity is achieved after about 1 to 2 or more weeks, or after about 1 to 2 months, of initiating the administration.

9. The method of claim 6, wherein the at least about 5% to 10% reduction in OCD symptom severity is maintained for at least about 4 to 8 weeks after discontinuing the administration.

10. The method of any one of the preceding claims, wherein at least one of said formulation, the pharmaceutical or the pharmaceutical preparation is formulated as a chewable delivery vehicle, a gum, a gummy, a candy, a lozenge, an ice cream or an ice, or a yogurt.

11. The method of any one of the preceding claims, wherein a unit dosage is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.

12. The method of any one of the preceding claims, wherein a daily dosage is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.

13. The method of any one of the preceding claims, wherein a unit dosage is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

14. The method of any one of the preceding claims, wherein the daily dosage is about 25 mg to grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.

15. The method of any one of the preceding claims, wherein the daily dosage is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week,
and optionally this “ramping up” or increasing of dosages continues for about a month, about 6 months or about a year, or until symptoms of OCD significantly diminish or abate, or significantly diminish or abate without need for administration of the formulation, the pharmaceutical or the pharmaceutical preparation.

16. The method of any one of the preceding claims, wherein at least one of said formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

17. The method of any one of the preceding claims, wherein at least one of said formulation, the pharmaceutical or the pharmaceutical preparation further comprises a preservative, a benzoic acid or a potassium sorbate.

18. The method of any one of the preceding claims, wherein at least one of said formulation, the pharmaceutical or the pharmaceutical preparation further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.

19. The method of any one of the preceding claims, wherein at least one of said formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch.

20. The method of any one of the preceding claims, wherein at least one of said formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

21. The method of any one of the preceding claims, wherein at least one of said formulation, the pharmaceutical or the pharmaceutical preparation further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

22. The method of any one of the preceding claims, wherein at least one of said formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

23. The method of any one of the preceding claims, wherein at least one of said formulation, the pharmaceutical or the pharmaceutical preparation is contained in a delivery vehicle, product of manufacture, container, syringe, device or bag.

24. The method of any one of the preceding claims, wherein at least one of said formulation, the pharmaceutical or the pharmaceutical preparation is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

25. The method of any one of the preceding claims, wherein the rifaximin, polymorphic form of a rifaximin, or rifaximin equivalent thereof, (optionally a XIFAXAN™, XIFAXANTA™ or NORMIX™) and the at least one additional antimicrobial or antibiotic agent are administered simultaneously.

26. The method of any one of the preceding claims, wherein the rifaximin, polymorphic form of a rifaximin, or rifaximin equivalent thereof, (optionally a XIFAXAN™, XIFAXANTA™ or NORMIX™) and the at least one additional antimicrobial or antibiotic agent are administered sequentially.

27. The method of any one of the preceding claims, comprising administering to the individual in need thereof rifaximin and a nitroimidazole.

28. The method of any one of the preceding claims, comprising administering to the individual in need thereof rifaximin and tinidazole.

29. The method of any one of the preceding claims, comprising administering to the individual in need thereof rifaximin, vancomycin, and metronidazole.
